(19) United States
(12) Patent Application Publication
(10) Pub. No.: US 2025/0275694 A1
(43) Pub. Date: Sep. 4, 2025

(54) BODY FLUID-BASED BIOLOGICAL DETECTION APPARATUS AND METHOD

(71) Applicant: Point Fit Technology Limited, Hong Kong (HK)

(72) Inventors: Chen Chen, Hong Kong (HK); Christopher Alfredo, Hong Kong (HK)

(21) Appl. No.: 18/594,939

(22) Filed: Mar. 4, 2024

(51) Int. Cl. A61B 5/1477; A61B 5/00; A61B 5/145; A61B 10/00

(52) U.S. Cl. CPC A61B 5/1477; A61B 5/14517; A61B 5/6832; A61B 10/0064; A61B 5/14546

(57) ABSTRACT

A body fluid-based biological detection apparatus and method are disclosed, the apparatus including a water filter layer, a molecularly imprinted polymer layer, and a flexible printed circuit board layer in sequence from the bottom to the top, the flexible printed circuit board layer being integrated with a biochemical sensor, where in one state, the water filter layer filters water from a body fluid, and the molecularly imprinted polymer layer recognizes and binds a biomarker in the filtered body fluid, and the binding of the biomarker to the molecularly imprinted polymer layer results in a change in electrical current of the biochemical sensor, so as to convert a biological signal in the body fluid into a physical signal. The present disclosure is a body fluid-based biological detection apparatus and method with high sensitivity and stable detection results.

### TECHNICAL FIELD

The present disclosure relates to the technical field of biomarker detection, and in particular to a body fluid-based biological detection apparatus and method.

### BACKGROUND

Biological body fluids refer to a variety of liquids secreted by a person, such as saliva, blood, urine, sputum, sweat, gastric juice, as well as semen, and leukorrhea, and so on. With the advancement of medicine, the detection of these body fluids can not only help the subject to find the diseased parts of the body, but also to detect the hidden diseases in advance.

At present, the most common biological body fluid sample is blood. However, the blood sample detection in the prior art has the following drawbacks: 1. the collection of blood samples is invasive; 2. blood samples need to be detected and analyzed in a laboratory, which inevitably places high demands on the detection environment and instrumentation; and 3. the detection is discontinuous.

In recent years, more and more researchers have been working on non-invasive, easy-to-collect, real-time, continuous biological body fluids as samples for health monitoring. The Chinese patent with Publication No. CN111671437A, entitled WEARABLE SWEAT DETECTION SYSTEM AND METHOD AND WEARABLE EQUIPMENT, discloses a wearable sweat detection system and method and wearable equipment, where the system includes: a microfluidic chip, an electrochemical electrode, a circuit acquisition system, and a mobile terminal system, where the microfluidic chip is provided with a sweat collection portion on a side fitting with the skin, and the microfluidic chip is provided with a sweat concentration cavity on the other side, the sweat concentration cavity being in contact with the electrochemical electrode; and the circuit acquisition system converts electrical signals of the sweat components as detected by the electrochemical electrode into digital signals and sends them to the mobile terminal system, and the mobile terminal system receives and analyzes and processes the digital signals and outputs the sweat detection result. The wearable sweat detection system and method and wearable equipment provided in this invention are easy to operate, and convenient to carry, and are capable of performing real-time collection, detection, analysis, and output of the sweat of the subject, so as to provide real-time monitoring and timely early warning of human body health state information. However, the detection system provided by this technical scheme suffers from the following drawbacks: 1. it does not check for biomarkers; and 2. microfluidics are used to collect sweat samples, which requires a certain amount of sweat.

In addition, the sweat detection can avoid the drawbacks of the blood detection, but the sweat detection apparatus of the prior art also suffers from defects such as low sensitivity and unstable detection results.

### SUMMARY

In view of the above problems, the present disclosure provides a body fluid-based biological detection apparatus and method with high sensitivity and stable detection results.

For this purpose, the present disclosure provides the following technical schemes:

A first aspect of the present disclosure provides a body fluid-based biological detection apparatus including a water filter layer, a molecularly imprinted polymer layer, and a flexible printed circuit board layer in sequence from the bottom to the top, the flexible printed circuit board layer being integrated with a biochemical sensor, where in one state, the water filter layer filters water from a body fluid, and the molecularly imprinted polymer layer recognizes and binds a biomarker in the filtered body fluid, and the binding of the biomarker to the molecularly imprinted polymer layer results in a change in electrical current of the biochemical sensor, so as to convert a biological signal in the body fluid into a physical signal.

In the present disclosure, the water filter layer filters water from the body fluid, such as sweat, in which water makes up about 99% while biomarkers make up a very low percentage. The present inventors, after extensive experimentation, have found that filtering the water in the sample solution prior to detection can increase the sensitivity and accuracy of the detection result.

In the present disclosure, the molecularly imprinted polymer layer can precisely bind to the biomarker that needs to be detected, thus enabling the detection result of the detection apparatus to be more accurate.

In the present disclosure, the flexible printed circuit board layer can be integrated with a variety of electronic components and can be functionally expanded according to the actual needs of the user.

In the present disclosure, the detection apparatus includes, but is not limited to, a water filter layer, a molecularly imprinted polymer layer, and a flexible printed circuit board layer in sequence from the bottom to the top. In actual use, a housing may be added outside the circuit board layer, or a water filter layer may be loaded outside the circuit board layer as protection for the electronic components of the circuit board layer.

Optionally, the flexible printed circuit board layer is further integrated with a humidity sensor, where in one state, the humidity sensor monitors the humidity around the detection apparatus and transmits humidity information in real time to the biochemical sensor, and the biochemical sensor analyzes and calculates the concentration of the biomarker taking the humidity data as a volumetric parameter of the body fluid and in combination with data of the biomarker transmitted by the molecularly imprinted polymer layer.

Optionally, the humidity sensor includes an MXene-based humidity sensor.

In the present disclosure, the humidity sensor may be any one or more kinds of small sensors, including, but not limited to, a MXene-based humidity sensor. The choice of a small sensor is to control the overall volume of the detection apparatus and to facilitate the use of the detection apparatus, especially in wearable or adhesive applications.

Optionally, the water filter layer includes a UHMWPE membrane.

Herein, the water filter layer may be any one or more compounds having a highly porous and highly permeable structure, and the water filter layer is selected as a green material that is non-toxic and non-hazardous to living organisms, based on the wearable or adhesive applications of the detection apparatus.

Optionally, the biochemical sensor includes a biochemical sensor based on organic electrochemical transistors.

Optionally, an intelligent terminal is further included, the intelligent terminal being connected to the flexible printed circuit board layer through a network signal; and further preferred, the intelligent terminal including a mobile phone, a computer, an iPad, or the like.

Optionally, the detection apparatus is wearable or adhesive, where further preferably, when the detection apparatus is wearable, the detection apparatus further includes a collar; and when the detection apparatus is adhesive, the detection apparatus does not require the use of an additional adhesive, but rather the detection apparatus relies only on van der Waals forces to adhere to the skin.

In the present disclosure, the detection apparatus is wearable or adhesive, which gives it the advantage of ease of use and good market prospects.

Optionally, the biomarker includes biological metabolites, hormones, electrolytes and proteins; and further preferred, the biological metabolites include but are not limited to lactic acid, uric acid, and glucose, the hormones include but are not limited to cortisol, the electrolytes include but are not limited to sodium, chloride, and hydrogen ions, and the proteins include but are not limited to C-reactive protein.

Optionally, the body fluid includes sweat and sweat vapor.

In the present disclosure, by detecting biomarkers in sweat vapor, interference of the detection result by unconscious sweating can be avoided.

Optionally, the thickness of the detection apparatus is 100-500 nm; and further preferably, the thickness of the detection apparatus is 150 nm or 200 nm or 250 nm or 300 nm or 350 nm or 400 nm or 450 nm.

Optionally, the detection apparatus further includes an early warning system which is written with a threshold, where the early warning system initiates an alarm when the result of detection of the biochemical sensor is above or below the threshold. Even further preferably, the early warning system is connected to an intelligent terminal of a hospital or a family doctor externally via a wireless network, and after the early warning system initiates an alarm, the intelligent terminal of the hospital or the family doctor acquires the early warning information in real time and intervenes in a timely manner.

A second aspect of the present disclosure provides a body fluid-based biological detection method, including the following steps:
at S1, a water filter layer filters water from a body fluid;
at S2, a molecularly imprinted polymer layer recognizes and binds a biomarker in the filtered body fluid;
at S3, the binding of the biomarker to the molecularly imprinted polymer layer results in a change in electrical current of the biochemical sensor, so as to convert a biological signal in the body fluid into a physical signal; and
at S4, the biochemical sensor analyzes the physical signal of the biomarker in the body fluid.

Optionally, the body fluid includes sweat and sweat vapor, the method further including the following steps: a detection result of a biomarker in sweat vapor in the case of unconscious sweating is used to correct a detection result of a biomarker in sweat of a subject.

Optionally, the detection method performs detection using a body fluid-based biological detection apparatus provided in the present disclosure, the detection apparatus including a water filter layer, a molecularly imprinted polymer layer, and a flexible printed circuit board layer in sequence from the bottom to the top, the flexible printed circuit board layer being integrated with a biochemical sensor, where in one state, the water filter layer filters water from a body fluid, and the molecularly imprinted polymer layer recognizes and binds a biomarker in the filtered body fluid, and the binding of the biomarker to the molecularly imprinted polymer layer results in a change in electrical current of the biochemical sensor, so as to convert a biological signal in the body fluid into a physical signal.

Optionally, the flexible printed circuit board layer is further integrated with a humidity sensor, where in one state, the humidity sensor monitors the humidity around the detection apparatus and transmits humidity information in real time to the biochemical sensor, and the biochemical sensor analyzes and calculates the concentration of the biomarker taking the humidity data as a volumetric parameter of the body fluid and in combination with data of the biomarker transmitted by the molecularly imprinted polymer layer.

Optionally, the humidity sensor includes an MXene-based humidity sensor.

Optionally, the water filter layer includes a UHMWPE membrane.

Optionally, the biochemical sensor includes a biochemical sensor based on organic electrochemical transistors.

Optionally, an intelligent terminal is further included, the intelligent terminal being connected to the flexible printed circuit board layer through a network signal.

Optionally, the detection apparatus is wearable or adhesive.

Optionally, the biomarker includes biological metabolites, hormones, electrolytes and proteins.

Optionally, the thickness of the detection apparatus is 100-500 nm.

The beneficial effects and significant progress of applying the technical schemes of the present disclosure compared to the prior art are as follows:
the body fluid-based biological detection apparatus of the present disclosure is thinner, which is 100-500 nm, and is thus more suitable for wearable or adhesive use;
the body fluid-based biological detection apparatus of the present disclosure can remove water from the body fluid by the water filter layer, while the biomarkers in the sweat will be retained in the molecularly imprinted polymer layer by the membrane and bound to the molecularly imprinted polymer layer, so that the biomarkers can be obtained more accurately through the molecularly imprinted polymer layer, which, ultimately, makes the detection apparatus more sensitive and accurate; in addition, most of the existing products in the market use the microfluidic mode to detect sweat, whereas the apparatus of the present disclosure can use sweat vapor for biomarker detection, so the detection result of the present disclosure is more accurate;
and the body fluid-based biological detection apparatus of the present disclosure has a newly added humidity sensor that can be used to measure the concentration of biomarkers, and the body fluid-based biological detection apparatus of the present disclosure can also be used to detect unconscious sweating, and by comparing data of the microbial markers in the sweat and the sweat vapor, interference of the data by unconscious sweating can be avoided.

### BRIEF DESCRIPTION OF DRAWINGS

In order to more clearly illustrate the technical schemes of the present disclosure, a brief description of the accompanying drawings required to be used in embodiments of the present disclosure will be given below.

Obviously, the accompanying drawings in the following description are only the accompanying drawings of some of the embodiments of the present disclosure, and for those of ordinary skill in the art, other accompanying drawings may be obtained based on these drawings without the expenditure of creative labor, but these other accompanying drawings are also within the accompanying drawings required to be used in the embodiments of the present disclosure.

FIG. 1 is a schematic diagram of a body fluid-based biological detection apparatus in Embodiment 1 of the present disclosure;

FIG. 2 is a partial schematic diagram of a body fluid-based biological detection apparatus in Embodiment 1 of the present disclosure;

FIG. 3 is a diagram of the use state of a body fluid detection apparatus of the prior art in Embodiment 2 of the present disclosure;

FIG. 4 is a microscopic view of the material of a body fluid-based biological detection apparatus in Embodiment 2 of the present disclosure;

FIG. 5 is a diagram of a skin-friendliness experiment of a body fluid-based biological detection apparatus in Embodiment 3 of the present disclosure;

FIG. 6 is a principle diagram of a body fluid-based biological detection apparatus in Embodiments 4 and 5 of the present disclosure; and

FIG. 7 is a flowchart of a body fluid-based biological detection apparatus in Embodiments 4 and 5 of the present disclosure.

List of reference numerals: 1. water filter layer; 2. molecularly imprinted polymer layer; 3. flexible printed circuit board layer; 3.1. biochemical sensor; 3.2. humidity sensor.

### DETAILED DESCRIPTION

The present disclosure is further explained below in conjunction with specific embodiments. The embodiments are intended to illustrate the present disclosure only and are not intended to limit the scope of the present disclosure. Furthermore, it should be understood that after reading the contents of the present disclosure, a person skilled in the art can make various alterations and modifications to the present disclosure, and these equivalent forms likewise fall within the scope limited by the claims appended to the present disclosure.

In order to more fully understand the present disclosure, specialized terms in the present disclosure are explained and described below.

A biochemical sensor, which may also be referred to as an electrochemical sensor, is a device or apparatus that is capable of sensing a biochemical quantity and converting it into a measurable physical signal (an optical signal, an electrical signal, or the like) in accordance with certain laws.

The biochemical sensor is a research field formed by the inter-penetration of multiple disciplines such as biology, chemistry, physics, electronics, medicine, semiconductor technology, and so on, which has the characteristics of good selection, high sensitivity, fast analysis, low cost, and so on, and can perform on-line continuous monitoring in a complex system, so that it is widely used in the fields of chemistry, life sciences, biomedicine, environmental monitoring, food, medicine, and the military, and so on. A biochemical sensor is structurally composed of two main parts: a sensitive membrane (a highly sensitive modified membrane) and a converter, where the sensitive membrane (a highly sensitive modified membrane) reacts in some way with the substance under measurement and can recognize the biological or chemical quantity under measurement, so as to convert the measured changes into some measurable changes through the sensitive membrane; and the converter can convert the measured signals sensed by the sensitive membrane into physical signals for easy measurement.

The molecularly imprinted polymer refers to the use of some natural compounds or synthetic compounds to mimic the biological system for molecular recognition studies, and the polymer synthesized by this molecular imprinting technique with specific recognition and selective adsorption is called the molecularly imprinted polymer. Molecular imprinting is achieved by the following methods: (1) imprinting molecules and functional monomers are bound by covalent bonds or II and non-covalent bonds to form a template-monomer complex; (2) a crosslinker is added to the complex, which is initiated by an initiator, heat or light, and a polymerization reaction occurs around the imprinting molecule-monomer complex. In this process, the polymer chain “captures” the template molecule and monomer complexes into the three-dimensional structure of the polymer by means of free radical polymerization; (3) the imprinting molecules in the polymer are extracted or dissociated by appropriate methods to form binding sites having recognized imprinting molecules.

As to MXene-based humidity sensors, MXene materials are a class of metal-carbide and metal-nitride materials with a two-dimensional layered structure, similar in appearance to potato chips stacked on top of each other. The chemical formula of the MXene material is Mₙ₊₁AXₙ, where (n=1-3), and M represents an early transition metal, such as Sc, Ti, Zr, V, Nb, Cr or Mo; A usually represents the third and fourth main group chemical elements; and X represents the element C or N. The MXene-based humidity sensor utilizes the excellent hydrophilicity and conductivity of MXene, in which MXene nanosheets are coated on chitosan-modified TPU electrostatically spun nanofibers by electrostatic interactions, and thereby a MXene/TPU composite film is prepared, and humidity sensors are prepared based on this film. A joint research team from the First Affiliated Hospital of Xi′an Jiaotong University (XJTU) and the School of Advanced Materials and Nanotechnology of Xi′an Electrical Science and Technology University (XESTU) has published a paper titled “MXene/TPU Composite Film for Humidity Sensing and Human Respiration Monitoring”. This research work utilizes the excellent hydrophilicity and conductivity of MXene, in which MXene nanosheets are coated on chitosan-modified TPU electrostatically spun nanofibers by electrostatic interactions, and thereby a MXene/TPU composite film is prepared, and humidity sensors are prepared based on this film. Based on the principle that the change of water molecule concentration affects the spacing between MXene nanosheets and thus changes the tunneling resistance, the MXene/TPU humidity sensor exhibits many characteristics, such as a high response speed (12 s), wide range of humidity response (11%-94% relative humidity (RH)), low hysteresis (<7% RH), and high reproducibility, and so on.

For a biochemical sensor based on organic electrochemical transistors, organic electrochemical transistors (OECTs) have the characteristic of high sensitivity. A sensor based on OECTs is usually controlled by two interfaces, namely gate/electrolyte and electrolyte/channel, and a change in either of the interfaces can cause a change in the performance of the device.

A UHMWPE membrane, which is made of ultra-high molecular weight polyethylene, or UHMWPE for short, has the characteristics of being low-cost, flexible, stretchable, and porous. Chinese patent CN113263747B discloses that a novel, robust, and mechanically flexible 100 nm HMWPE membrane with a polygonal pore structure can be prepared in an easily scalable manner using an initially low entanglement UHMWPE membrane. With a tensile strength of up to 900 MPa and a ductility of 26%, this novel nanofilm can be widely applied in many important technological fields.

### Embodiment 1

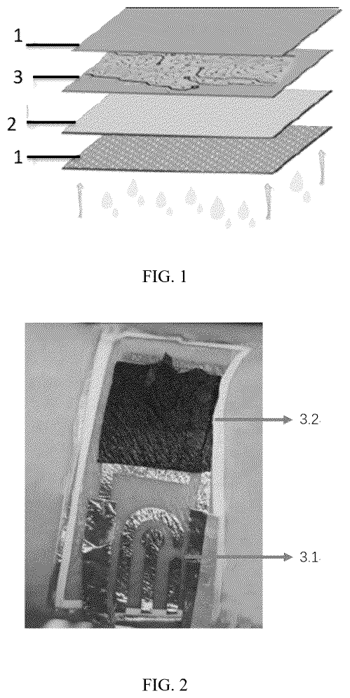

As shown in FIG. 1, a body fluid-based biological detection apparatus includes a water filter layer 1, a molecularly imprinted polymer layer 2, and a flexible printed circuit board layer 3 in sequence from the bottom to the top, the flexible printed circuit board layer 3 being integrated with a biochemical sensor 3.1, where the water filter layer 1 filters water from a body fluid, and the molecularly imprinted polymer layer 2 recognizes and binds a biomarker in the filtered body fluid, and the binding of the biomarker to the molecularly imprinted polymer layer 2 results in a change in electrical current of the biochemical sensor 3.1, so as to convert a biological signal in the body fluid into a physical signal.

As shown in FIG. 2, the flexible printed circuit board layer 3 is further integrated with a humidity sensor 3.2, where the humidity sensor 3.2 monitors the humidity around the detection apparatus and transmits humidity information in real time to the biochemical sensor 3.1, and the biochemical sensor 3.1 analyzes and calculates the concentration of the biomarker taking the humidity data as a volumetric parameter of the body fluid and in combination with data of the biomarker transmitted by the molecularly imprinted polymer layer 2.

In this embodiment, the humidity sensor includes an MXene-based humidity sensor.

In this embodiment, the water filter layer includes a UHMWPE membrane.

In this embodiment, the biochemical sensor includes a biochemical sensor based on organic electrochemical transistors.

In this embodiment, an intelligent terminal is further included, the intelligent terminal being connected to the flexible printed circuit board layer through a network signal.

In this embodiment, the detection apparatus is adhesive

In this embodiment, the biomarker includes biological metabolites, hormones, electrolytes and proteins.

In this embodiment, the body fluid includes sweat and sweat vapor.

In this embodiment, the thickness of the detection apparatus is 150 nm.

### Embodiment 2

A sweat detection comparison experiment was conducted between the body fluid-based biological detection apparatus of Embodiment 1 (experimental group) and a commercially available sweat detection apparatus (control group). The apparatuses of the experimental and control groups were adhered to the right and left arms of the subject, and the time of the first detection of biological signals, the total amount of biological signals detected, and the skin condition around the apparatuses of the experimental and control groups were counted separately.

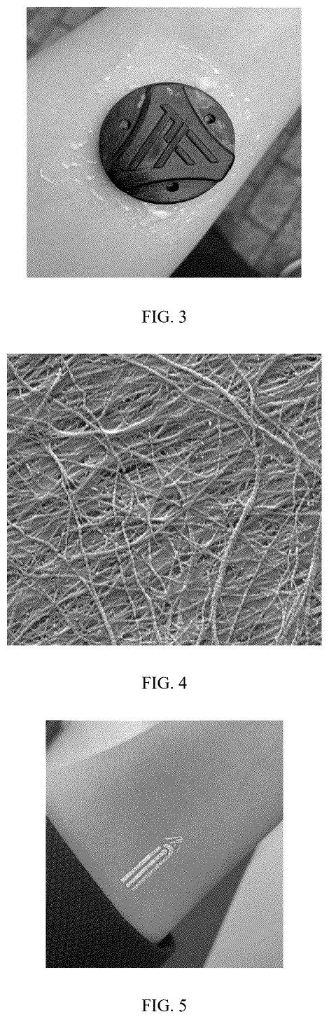

The detection result was a large number of visible air bubbles around the commercially available sweat detection apparatus, as shown in FIG. 3, which may be attributed to the fact that the water from the sweat was trapped at that location. In contrast, there were no visible air bubbles around the body fluid-based biological detection apparatus of Embodiment 1. Further, the water filter layer of the body fluid-based biological detection apparatus of Embodiment 1 was observed under a microscope, and the detection result, as shown in FIG. 4, showed that there were a large number of voids in the water filter layer, and it is possible that the voids in the water filter layer increase the evaporation of water. Moreover, this significantly avoids interference from sweat from other regions (non-detection regions), thus ensuring cleanliness around the apparatus. In addition, the experimental group also had a shorter time to first obtain a biological signal and obtained more biological signals than the control group. By analyzing the reason, it can be seen that this may be due to the fact that the water filter layer in the experimental group filtered out most of the water in the sweat, resulting in a greater concentration of biomarkers in the samples tested. Moreover, the molecularly imprinted polymer layer in the experimental group can accurately recognize biomarkers.

### Embodiment 3 Skin-Friendliness Experiment

The preparation material for the water filter layer of the body fluid-based biological detection apparatus of Embodiment 1 was applied to the surface of the skin of the subject, and the surface of the skin was observed for a period of time, and the feelings of the subject were recorded.

The result is as shown in FIG. 5. There was no redness, swelling, or blotchy rash on the skin of the subject, and the subject did not feel uncomfortable.

### Embodiment 4

This embodiment uses the body fluid-based biological detection apparatus of Embodiment 1 for sweat detection and analysis.

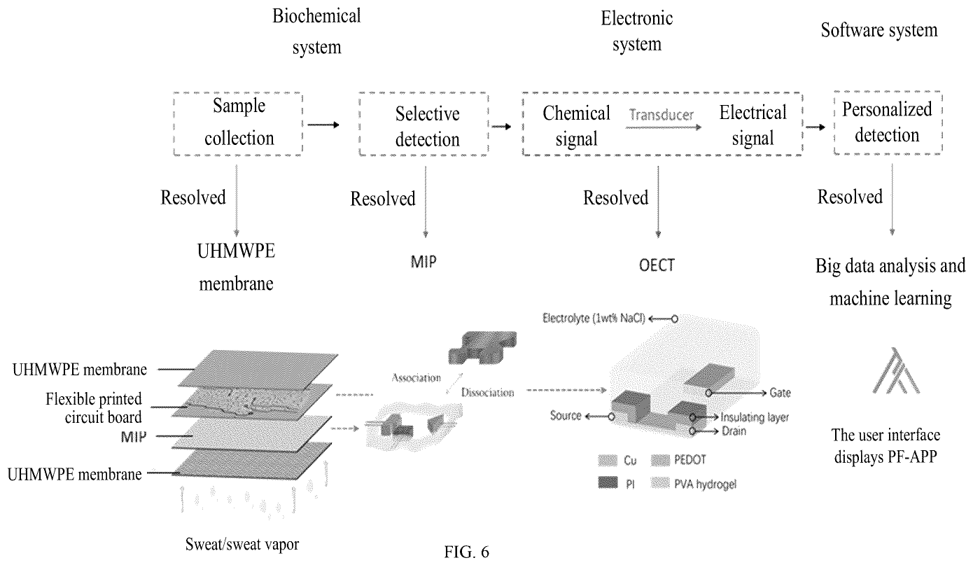

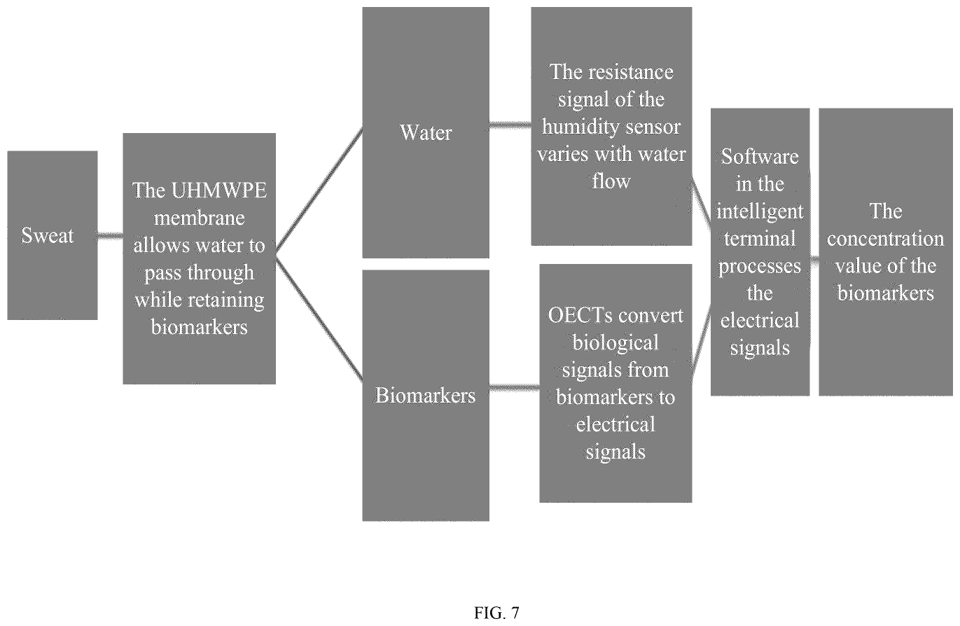

As shown in FIGS. 6 and 7, it mainly includes the following steps:
At step I, the first step in analyzing any biofluid (body fluid) is to correctly collect a detection sample from the subject's body, which is critical because it is difficult to accurately quantify a biomarker if the detection sample is not correctly collected. In particular, sweat contains 99% water and only 1% biomarkers, which makes correct collection even more important. Therefore, a highly porous and breathable material (UHMWPE membrane water filter layer) is used, which enables rapid evaporation of water, and retains only biomarkers in the ultra-high molecular weight polyethylene membrane;
At step II, however, the required biomarkers may make up only a small fraction (e.g., 0.1%) of all the biomarkers, so there is a need for deeper molecular recognition capabilities. For our example, a molecularly imprinted polymer (MIP) is added, which specifically binds the desired molecules and is then connected to the organic electrochemical transistors (OECTs) of the biochemical sensor;
At step III, biological signals are converted to electrical signals that flow in the OECTs, and biochemical sensors detect and analyze the electrical signals;
At step IV, in addition, in order to capture the volume of collected sweat for each user's calibration, humidity measurements were made using a MXene-based sensor that correlates changes in resistance with the collected sweat vapor, which enables the combination of the collected biomarkers and the volume so as to obtain an accurate concentration of sweat biomarkers; and
At step V, finally, after real-time tracking of biomarkers is achieved, a software system is developed, which can process and build measurements of each person providing personalized algorithms.

### Embodiment 5

This embodiment uses the body fluid-based biological detection apparatus of Embodiment 1 for detection and analysis of sweat and sweat vapor.

As shown in FIGS. 6 and 7, it mainly includes the following steps:
At step I, the first step in analyzing any biofluid (body fluid) is to correctly collect a detection sample from the subject's body, which is critical because it is difficult to accurately quantify a biomarker if the detection sample is not correctly collected. In particular, sweat contains 99% water and only 1% biomarkers, which makes correct collection even more important. Therefore, a highly porous and breathable material (UHMWPE membrane water filter layer) is used, which enables rapid evaporation of water, and retains only biomarkers in the ultra-high molecular weight polyethylene membrane;
At step II, however, the required biomarkers may make up only a small fraction (e.g., 0.1%) of all the biomarkers, so there is a need for deeper molecular recognition capabilities. For our example, a molecularly imprinted polymer (MIP) is added, which specifically binds the desired molecules and is then connected to the organic electrochemical transistors (OECTs) of the biochemical sensor;
At step III, biological signals are converted to electrical signals that flow in the OECTs, and biochemical sensors detect and analyze the electrical signals;
At step IV, in addition, in order to capture the volume of collected sweat for each user's calibration, humidity measurements were made using a MXene-based sensor that correlates changes in resistance with the collected sweat vapor, which enables the combination of the collected biomarkers and the volume so as to obtain an accurate concentration of sweat biomarkers;
At step V, finally, after real-time tracking of biomarkers is achieved, a software system is developed which can process and build measurements of each person providing personalized algorithms;
At step VI, biomarkers are detected in the subject's unconscious sweating (sweat vapor) using the method of steps I-V; and
At step VII, comparison and analysis of the data obtained at step V with the data obtained at step VI is performed.

The applicant declares that during the description of the above specification:

Descriptions of the terms “this embodiment”, “an embodiment of the present disclosure”, “as shown in . . . ”, “further”, “further improved technical sub-scheme”, or the like, mean that specific features, structures, materials or features described in the embodiment or example are included in at least one embodiment or example of the present disclosure; in this specification, the schematic formulation of the above terms is not necessarily directed to the same embodiments or examples, and the specific features, structures, materials, characteristics, or the like, described may be integrated or combined in an appropriate manner in any one or more embodiments or examples; in addition, those of ordinary skill in the art can integrate or combine different embodiments or examples and features of different embodiments or examples described in the present specification, provided that no contradiction arises.

Finally, it should be noted that
the above embodiments are intended only to illustrate the technical schemes of the present disclosure and are not intended to be a limitation thereof;
although the present disclosure has been described in detail with reference to the foregoing embodiments, those of ordinary skill in the art should understand that it is still possible for them to make modifications to the technical schemes recorded in the foregoing embodiments, or to make equivalent replacements for some or all of the technical features therein, and such modifications or replacements do not cause the essence of the corresponding technical schemes to depart from the scope of the technical schemes of the embodiments of the present disclosure, and all non-essential improvements and adjustments or replacements made by a person skilled in the art based on the contents of this specification are within the scope of the claimed protection of the present disclosure.

1. A body fluid-based biological detection apparatus, comprising a water filter layer, a molecularly imprinted polymer layer, and a flexible printed circuit board layer in sequence from the bottom to the top, the flexible printed circuit board layer being integrated with a biochemical sensor, wherein in one state, the water filter layer filters water from a body fluid, and the molecularly imprinted polymer layer recognizes and binds a biomarker in the filtered body fluid, and the binding of the biomarker to the molecularly imprinted polymer layer results in a change in electrical current of the biochemical sensor, so as to convert a biological signal in the body fluid into a physical signal.

2. The body fluid-based biological detection apparatus of claim 1, wherein the flexible printed circuit board layer is further integrated with a humidity sensor, wherein
in one state, the humidity sensor monitors the humidity around the detection apparatus and transmits humidity information in real time to the biochemical sensor, and the biochemical sensor analyzes and calculates the concentration of the biomarker taking the humidity data as a volumetric parameter of the body fluid and in combination with data of the biomarker transmitted by the molecularly imprinted polymer layer.

3. The body fluid-based biological detection apparatus of claim 2, wherein the humidity sensor comprises an MXene-based humidity sensor.

4. The body fluid-based biological detection apparatus of claim 1, wherein the water filter layer comprises a UHMWPE membrane.

5. The body fluid-based biological detection apparatus of claim 1, wherein the biochemical sensor comprises a biochemical sensor based on organic electrochemical transistors.

6. The body fluid-based biological detection apparatus of claim 1, further comprising an intelligent terminal, the intelligent terminal being connected to the flexible printed circuit board layer through a network signal.

7. The body fluid-based biological detection apparatus of claim 1, wherein the detection apparatus is wearable or adhesive.

8. The body fluid-based biological detection apparatus of claim 1, wherein the biomarker comprises biological metabolites, hormones, electrolytes and proteins.

9. The body fluid-based biological detection apparatus of claim 7, wherein the body fluid comprises sweat and sweat vapor.

10. The body fluid-based biological detection apparatus of claim 1, wherein the thickness of the detection apparatus is 100-500 nm.

11. A body fluid-based biological detection method, comprising the following steps:
at S1, a water filter layer filters water from a body fluid;
at S2, a molecularly imprinted polymer layer recognizes and binds a biomarker in the filtered body fluid;
at S3, the binding of the biomarker to the molecularly imprinted polymer layer results in a change in electrical current of the biochemical sensor, so as to convert a biological signal in the body fluid into a physical signal; and
at S4, the biochemical sensor analyzes the physical signal of the biomarker in the body fluid.

12. The body fluid-based biological detection method of claim 11, wherein the body fluid comprises sweat and sweat vapor, the method further comprising the following steps: a detection result of a biomarker in sweat vapor in the case of unconscious sweating is used to correct a detection result of a biomarker in sweat of a subject.
